(19) United States
(12) Patent Application Publication
(10) Pub. No.: US 2013/0317109 A1
(43) Pub. Date: Nov. 28, 2013

(54) PROCESS FOR THE PREPARATION OF LACOSAMIDE

(71) Applicants: Cecilia Kvarnström Branneby, Karlskoga (SE); Margus Eek, Tallinn (EE)

(72) Inventors: Cecilia Kvarnström Branneby, Karlskoga (SE); Margus Eek, Tallinn (EE)

(73) Assignee: CAMBREX KARLSKOGA AB, Karlskoga (SE)

(21) Appl. No.: 13/989,215

(22) PCT Filed: Nov. 25, 2011
(86) PCT No.: PCT/GB2011/052339

§ 371 Date: Aug. 8, 2013

Related U.S. Application Data
(60) Provisional application No. 61/436,251, filed on Jan. 26, 2011.

(30) Foreign Application Priority Data
Nov. 25, 2010 (GB) 1020026.9

(51) Int. Cl. C12P 13/02; C07C 245/24; C07C 231/12

(52) U.S. Cl. CPC C12P 13/02; C07C 231/12; C07C 245/24
USPC 514/616; 564/196; 552/12; 435/129

(57) ABSTRACT

There is provided a process for the preparation of Lacosamide (which is a useful medicament) of formula I, which comprises an enantioselective enzymatic acylation.

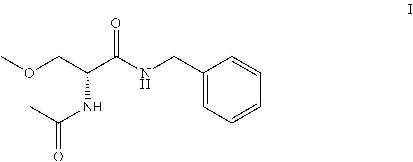 [SMILES: COC[C@@H](NC(C)=O)C(=O)NCc1ccccc1]

The present invention relates to a new resolution process for the preparation of an enantiomerically enriched amide, in particular for the preparation of the single enantiomer Lacosamide (which is the (R)-enantiomer) from the corresponding amino precursor.

Lacosamide is an anti-convulsive drug, useful for the adjunctive treatment of partial onset seizures and diabetic neuropathic pain.

Processes for preparing Lacosamide have been disclosed in international patent application WO 2010/052011, as well as in earlier documents including US patent documents U.S. Pat. No. 5,378,729, U.S. Pat. No. 5,773,475, U.S. Pat. No. 6,048,899 and US 2008/0027137, European patent documents EP 1 642 889 and EP 2 067 765 and Chinese patent document CN 101591300.

Earlier processes usually employ D-serine as a starting material, which is expensive and therefore has a drawback. The more recent process described in international patent application WO 2010/052011 discloses a resolution of Lacosamide (i.e. the amide), which resolution step is performed by the use of certain chiral chromatographic techniques. The undesired amide enantiomer is then racemised in a separate step, and the resolution to separate Lacosamide from the undesired amide enantiomer is repeated.

Certain dynamic kinetic resolutions are known in the art. For instance, U.S. Pat. No. 6,335,187 (and equivalent application WO 99/31264) discloses a process of resolution of chiral amines, by reacting an enantiomer of the amine with an alkyl ester in the presence of an enantioselective lipase enzyme to produce an enantiomer of the amide so formed, and separating it from the unreacted (amine) enantiomer. However, this document only relates to certain amine.

There is a need for alternative and/or improved reactions for the formation of single amide enantiomers (e.g. Lacosamide), which are more selective and/or advantageous in terms of being obtainable in higher yields and fewer (or less cumbersome) synthetic steps. This is important for process chemistry, in particular when scaling up.

The listing or discussion of an apparently prior-published document in this specification should not necessarily be taken as an acknowledgement that the document is part of the state of the art or common general knowledge.

There is now provided a process for the preparation of Lacosamide (formula I):

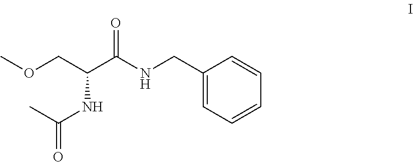 [SMILES: COC[C@@H](NC(C)=O)C(=O)NCc1ccccc1]

which process comprises a selective enzymatic acylation of a precursor of formula II,

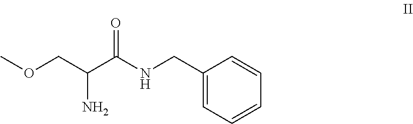

optionally further characterised in that the reaction is performed in the presence of a racemisation promoter,
which process is hereinafter referred to as “the process of the invention”.

The process of the invention comprises (as a first step) a selective enzymatic acylation, by which we refer to an acylation in the presence of an enzyme. The enzyme is any suitable enzyme that affects the selective (or enantioselective) conversion, for instance a suitable enantioselective hydrolase, e.g. lipase esterase or protease enzyme (or mixtures thereof). However, it will be appreciated by the skilled person that every possible enzyme in the foregoing list may not achieve the appropriate enantioselectivity, but this is something that the skilled person may be able to determine by trialling the appropriate enzyme (e.g. enzymes that are synthesised or those that are commercially available). However, in a particularly preferred embodiment, the enzyme that is employed is preferably selected from lipase B from Candida antarctica (CALB).

The compound of formula II is racemic, and has a chiral carbon atom that bears the —NH₂ group. Hence, the compound of formula II normally exists as two enantiomers in equal proportions; i.e. a racemate. However, if the compound of formula II is enantiomerically enriched (in any enantiomeric excess (ee), e.g. in slight ee), then this should not affect the process of the invention (i.e. the process of the invention will still promote the formation of the correct enantiomer of the compound of formula I, whilst racemising the undesired enantiomer and promoting further formation of the correct enantiomer).

The enzymatic acylation (i.e. reaction in the presence of enzyme) allows the formation of substantially one enantiomer of the amide of formula I (i.e. it allows selective reaction with only one of the amine enantiomers of the precursor of formula II), whilst leaving substantially all of the other undesired enantiomer unreacted (as the free amine).

The acylation (which includes “acetylation”) reaction clearly involves the presence of an acyl donor (i.e. a group that donates a —C(O)—CH₃ group to the amine, so forming an amide in an acylation reaction). The acyl donor may for instance be H₃C—C(O)-LG, in which LG is a suitable leaving group and hence the acyl donor may be:
(a) any alkyl acetate, e.g. a C₁₋₁₂ alkyl acetate (H₃C—C(O)—O—C₁₋₁₂ alkyl), in which the C₁₋₁₂ alkyl moiety may be cyclic or, preferably, linear or branched e.g. ethyl, n-propyl, isopropyl, n-butyl, n-hexyl, 2-ethylhexyl and vinyl (the alkyl group may also be optionally substituted, but is preferably unsubstituted). The most preferred C₁₋₁₂ alkyl group is a branched C₃₋₈ (e.g. C₃₋₄) alkyl group, e.g. isopropyl, and hence the most preferred acyl donor is isopropyl acetate;
(b) an acyl halide, e.g. H₃C—C(O)-L^{x}, in which L^{x} is halo (e.g. iodo or, preferably, chloro or bromo);
(c) an acyl amide, e.g. H₃C—C(O)—N(R¹⁰)R¹¹, in which R¹⁰ and R¹¹ independently represent hydrogen or optionally substituted C₁₋₁₂ alkyl;
(d) an anhydride (carboxylic acid anhydride), e.g. H₃—C(O)—O—C(O)—R¹², in which R¹² is optionally substituted C₁₋₁₂ alkyl (and preferably unsubstituted methyl, i.e. so that the anhydride is symmetrical).

Embodiments of the invention that may be mentioned therefore include those in which the acyl donor may be:
(a) any alkyl acetate, e.g. a C₁₋₁₂ alkyl acetate (H₃C—C(O)—O—C₁₋₁₂ alkyl), in which the C₁₋₁₂ alkyl moiety may be cyclic or, preferably, linear or branched e.g. ethyl, n-propyl, isopropyl, n-butyl, n-hexyl, 2-ethylhexyl and vinyl (the alkyl group may also be optionally substituted, but is preferably unsubstituted). The most preferred C₁₋₁₂ alkyl group is a branched C₃₋₈ (e.g. C₃₋₄ alkyl group, e.g. isopropyl, and hence the most preferred acyl donor is isopropyl acetate; or
(b) an acyl amide, e.g. H₃C—C(O)—N(R¹⁰)R¹¹, in which R¹⁰ and R¹¹ independently represent hydrogen or optionally substituted C₁₋₁₂ alkyl;

Further embodiments of the invention that may be mentioned therefore include those in which the acyl donor is a C₁₋₁₂ alkyl acetate (e.g. C₁₋₈ or C₁₋₆ alkyl acetate, particularly a branched C₃₋₈, or particularly, a branched C₃₋₄ alkyl acetate, such as isopropyl acetate).

By “selective” we mean that the enzyme promotes the acylation of substantially one enantiomer in favour of the other (i.e. it is an enantioselective reaction step). This includes any bias toward one of the two enantiomers, i.e. a ratio of reactivity of greater than 50:50 selectivity. However, clearly, in order to attain as much of the desired compound of formula I (in favour of the opposite enantiomer), the ratio of reactivity of the desired enantiomer to the undesired one is greater than 70:30 selectivity, preferably greater than 80:20 (e.g. 90:10) and most preferably, it is greater than 95:5 (e.g. greater than 97:3, most preferably at or near 100:0), which advantageously results in the most enantioselective formation of the product of formula I.

A single enantiomer (the (R)-enantiomer) of the compound of formula I is formed (i.e. Lacosamide). As the reaction is enantioselective, it is not dependent on the proportions of the two enantiomers of the starting material, which is usually a racemic mixture of the compound of formula II. By “single enantiomer” (or enantiomerically enriched compound) so formed, we mean that the enantiomeric excess of the product of formula I is greater than 0% (e.g. greater than 50%), i.e. there is more of one enantiomer than the other. Preferably, we mean that the enantiomeric excess is greater than 60%, more preferably greater than 70%. Particularly preferred are enantiomeric excesses greater than 80%, especially greater than 90%. Most preferably, the enantiomeric excess is close to 100% (i.e. greater than 95%, for example greater than 99%), with a negligible amount of the minor enantiomer.

The process of the invention therefore leaves (undesired) enantiomer of formula II unreacted (specifically the (S)-enantiomer, i.e. (S)-2-amino-N-benzyl-3-methoxypropaneamide) in the reaction mixture. Advantageously, the process of the invention is performed in the presence of a racemisation promoter (also referred to herein as “racemiser”), which converts the unreacted (and undesired) amine back to the racemic starting material, i.e. to a mixture of the (R) and (S) enantiomers. This allows the conversion of further material to the desired ((R)-enantiomer) product of formula I. This process may continue ad infinitum and hence provide (in principle) conversion of all, or substantially all, of the starting material into desired product. This is depicted in the Scheme 1 below.

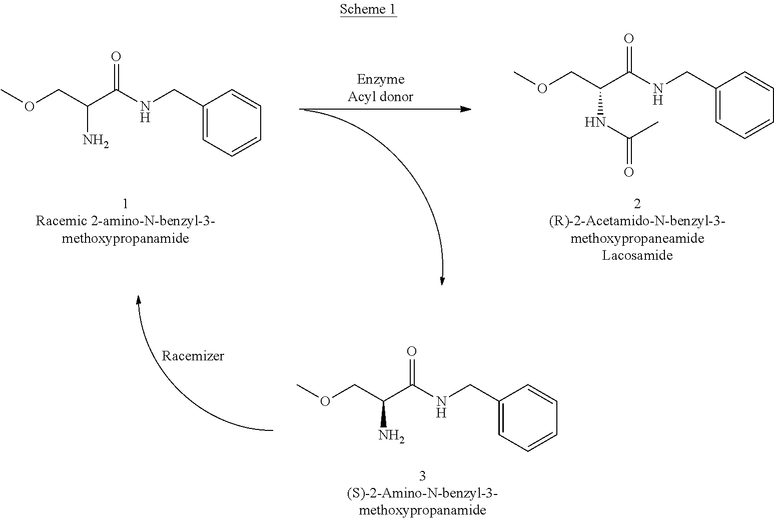 [SMILES: COCC(N)C(=O)NCc1ccccc1 ; COC[C@@H](NC(C)=O)C(=O)NCc1ccccc1 ; COC[C@H](N)C(=O)NCc1ccccc1]

The process of the invention is therefore a dynamic kinetic resolution, which is advantageous to any known resolutions for the preparation of Lacosamide, which may require separation (and/or isolation) of the undesired enantiomer resulting in a maximum yield of 50%. For instance, the process described in WO 2010/052011 described a resolution of the two enantiomers of racemic Lacosamide. Clearly, only a 50% yield is obtainable in this resolution step. In this instance, the undesired (S)-enantiomer of Lacosamide would have to be separated and racemised in a separate step (e.g. that involves hydrolysis of the amide and subsequent re-acylation) for the further resolution to take place. Hence, in a preferred embodiment, the dynamic kinetic resolution of the compound of the invention takes place in “one pot”. By this, we mean that, in the resolution step any undesired enantiomer (of starting material and/or product) need not be separated (and optionally recycled), but rather, in the process of the invention, the separation of the undesired enantiomer (of starting material) is circumvented by its conversion to the racemate in the reaction pot (thereby allowing further selective acylation, etc).

The racemisation promoter may be any suitable aldehyde, ketone or metal catalyst (but preferably, it is an aldehyde). This may promote or cause the racemisation by undergoing a reversible condensation reaction, i.e. starting with a single enantiomer (or enantiomerically enriched compound) of the compound of formula II (the undesired (S)-enantiomer) and then forming a racemic mixture of the compound of formula II (or a compound of lower ee), such that there is more of the desired enantiomer ((R)-enantiomer) that may undergo the enantioselective acylation reaction to form the single (R)-enantiomer product of formula I. The racemisation promoter (e.g. when it is a metal catalyst) may also promote or cause the racemisation by catalysing an oxidation-reduction reaction on the non-reacting amine (i.e. the (S)-enantiomer of the amine of formula II that does not acylate during the process of the invention; e.g. involving the corresponding imine derivative). The metal catalyst system may be any suitable one that promotes the appropriate reaction (e.g. by catalyzing the oxidation-reduction) to effect the racemisation. For instance, preferably, the metal catalyst is a precious metal (e.g. palladium) on carbon.

However, the racemisation promoter is preferably an aldehyde R¹—CHO (or the ketone R¹—C(O)—R²), in which each R¹ (and, independently, R²) may represent optionally substituted C₁₋₁₂ alkyl, but each R¹ (and, independently, R²) preferably represents an optionally substituted (i.e. contain one or more optional substituents) aryl/heteroaryl group (e.g. a monocyclic aryl or monocyclic 5- or 6-membered heteroaryl group, e.g. phenyl, pyridyl and the like), in which the optional substituents are preferably selected from: T¹ or C₁₋₁₂ alkyl optionally substituted by one or more substituents selected from T²; in which:

T¹ and T² are independently selected from halo, —NO₂, —CN, —C(O)₂R^{x1}, —OR^{x2}, —SR^{x3}, —S(O)R^{x4}, —S(O)₂R^{x5}, —N(R^{x6})R^{x7}, —N(R^{x8})C(O)R^{x9}, —N(R^{x10})S(O)₂R^{x11}, —O—P(O)(OR^{x12})(OR^{x13}) or R^{x14};
R^{x1}, R^{x2}, R^{x3}, R^{x6}, R^{x7}, R^{x8}, R^{x9}, R^{x10}, R^{x12} and R^{x13} independently represent hydrogen or C₁₋₆ alkyl optionally substituted by one or more halo atoms;
R^{x4}, R^{x5}, R^{x11} and R^{x14} independently represent C₁₋₆ alkyl optionally substituted by one or more halo atoms.

Preferred racemisation promoters include optionally substituted salicylic aldehyde, for instance unsubstituted salicylic aldehyde, pyridoxal-5′-phosphate (also referred to herein as “PLP”), dichlorosalicylic aldehyde (e.g. 3,5-dichlorosalicylic aldehyde), 5-nitrosalicylic aldehyde, nitro- or dinitro-benzaldehyde (e.g. 2-nitro, 4-nitro or 2,4-dinitro-benzaldehyde). Particularly preferred are salicylic aldehyde and pyridoxal-5′-phosphate. Those that are particularly advantageous include those that retain or do not substantially reduce the acylation/acetylation rate, and these include 5-nitrosalicylic aldehyde and 3,5-dichlorosalicylic aldehyde. Embodiments of the invention that may be mentioned therefore include those in which the racemisation promoter is dichlorosalicylic aldehyde or, particularly 5-nitrosalicylic aldehyde).

Racemisation using a racemisation promoter can be enhanced by adding a racemisation promoter activator, such as a base. A reduction in the amount of racemisation promoter that is required may then be possible.

Preferred racemisation promoter activators include inorganic bases (such as Na₂CO₃) or, preferably, amines (such as triethylamine (TEA), dimethylaminopyridine (DMAP), piperidine, methylpiperidine, or more preferably N,N,N′,N′-tetramethylethylenediamine (TMEDA)). The base may provided for, at least in part, by increasing the amount present of the compound of formula II that is to be racemised. The use of lower quantities of racemisation promoter in the reaction mixture is advantageous in that, for example, greater yields can be obtained following purification.

The racemisation promoter activator (e.g. an amine (such as TEA, DMAP, piperidine, methylpiperidine, or preferably TMEDA)) may be added in any suitable quantity, for instance from about 1 to about 50 mol %, based on the quantity of the compound of formula II. Preferably from about 2 to about 30 mol % (e.g. from about 5 to about 20 mol %) of the racemisation promoter is employed.

Unless otherwise specified, the process of the invention may be performed employing salts, solvates or protected derivatives, thereby producing compounds that may or may not be produced in the form of a (e.g. corresponding) salt or solvate, or a protected derivative thereof.

Unless otherwise specified, alkyl groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl groups may also be part cyclic/acyclic. Such alkyl groups may also be saturated or, when there is a sufficient number (i.e. a minimum of two) of carbon atoms, be unsaturated.

The term “aryl”, when used herein, includes C₆₋₁₀ groups. Such groups may be monocyclic, bicyclic or tricyclic and, when polycyclic, be either wholly or partly aromatic. C₆₋₁₀ aryl groups that may be mentioned include phenyl, naphthyl, and the like. For the avoidance of doubt, the point of attachment of substituents on aryl groups may be via any carbon atom of the ring system.

The term “heteroaryl”, when used herein, includes 5- to 14-membered heteroaryl groups containing one or more heteroatoms selected from oxygen, nitrogen and/or sulfur. Such heteroaryl group may comprise one, two or three rings, of which at least one is aromatic. Substituents on heteroaryl groups may, where appropriate, be located on any atom in the ring system including a heteroatom.

The point of attachment of heteroaryl groups may be via any atom in the ring system including (where appropriate) a heteroatom. Examples of heteroaryl groups that may be mentioned include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrinnidinyl, indolyl, pyrazinyl, indazolyl, pyrimidinyl, quinolinyl, benzoimidazolyl and benzthiazolyl.

The term “halo”, when used herein, includes fluoro, chloro, bromo and iodo.

The process of the invention is performed in the presence of a suitable enzyme. The enzyme may be immobilised on solid support, but may also be non-immobilised. In this respect, any suitable quantity of the enzyme may be employed in order to achieve the selective acylation. Typically, immobilised enzyme is employed, which may contain 1-5% (w/w) enzyme on the carrier (where the amounts are weights of enzyme+carrier). The process of the invention will still proceed if more than 100% of the enzyme (plus carrier, if present/employed) by weight of the compound of formula II is employed, even though this may be impractical. However, less than about 50% of the enzyme (plus carrier, if present/employed) by weight of the compound of formula II is employed. Most preferably, the amount of enzyme (plus carrier, if present) employed is from about 10% to about 50% (e.g. between about 10% and about 50%), particularly from about 20% to about 30% (e.g. between about 20 and 30%, more particularly about 25%) by weight of the compound of formula II. However, less than 10% may also be employed, e.g. less than 5% and even as low as about 1% of the enzyme (plus carrier, if present/employed) by weight of the compound of formula II.

The process of the invention may be performed in any suitable solvent (for example an organic solvent (such as THF or, particularly, 2-propanol) or a mixture of organic solvents). However, advantageously, the solvent that is employed may be the acyl donor, i.e. the acyl donor may serve as the solvent, without the need for an additional solvent. Hence the process of the invention may be performed in the presence of an alkyl acetate (e.g. a C₁₋₁₂, C₁₋₈, C₁₋₆ or branched C₃₋₈ (such as a branched C₃₋₄ alkyl acetate, such as isopropyl acetate).

The solubility of lacosamide in solvents such as isopropyl acetate may be improved by performing the process in the presence of a co-solvent. Co-solvents which may be used, particularly in conjunction with isopropyl acetate, include DMF, DMAA (N,N-dimethylacetamide), N-methylpyrrolidone (NMP), 2-propanol or, particularly, an ether, such as 2-methyl THF, methyl-tert-butyl ether (MTBE) or particularly, THF. When isopropyl acetate is used as the solvent, the co-solvent:solvent ratio is typically from about 10:1 to about 1:10, preferably from about 2:1 to about 1:5.

The process of the invention may be performed at room temperature, but may be performed at elevated temperature (e.g. from room temperature to about 100° C. or up to about 70° C.). This will depend on the solvent system employed in the process of the invention and the boiling point thereof, as well as on the tolerability of the enzyme to high temperatures. Preferably however (e.g. when isopropyl acetate is employed as the solvent), the process of the invention is performed at elevated temperature (e.g. at above 30° C., for instance at anything from about 30° C. to about 100° C., e.g. between about 30° C. and about 100° C. (particularly from about 35° C. to about 95° C. e.g. between about 35° C. and 95° C.; in an embodiment the preferred range is from about 50° C. to about 80° C. (e.g. between about 50 and 80° C.)).

The racemisation promoter (e.g. aldehyde as defined herein) may be added in any suitable quantity, for instance from about 0.1 to about 50 mol %, or, particularly, between about 1 and 50 mol %, based on the quantity of the compound of formula II.

Particular embodiments of the invention that may be mentioned include those in which the racemisation promoter is used at a concentration of from about 2 to about 20 mol % (or from about 5 to about 10 mol %) relative to the compound of formula II, or between about 2 and 20 mol % (e.g. between 5 and 10 mol %) relative to the compound of formula II.

The racemisation of 2-amino-N-benzyl-3-methoxypropionamide (S-amine) from racemic 2-amino-N-benzyl-3-methoxypropionamide can be achieved using a racemisation promoter, as indicated above, present in the amounts indicated above.

Racemisation can be achieved using lower concentrations (such as from about 0.1 to about 3 mol % (e.g. 3 mol % or less, such as from 1 to 2 mol %) relative to the compound of formula II) of the racemisation promoter by adding a racemisation promoter activator. The racemisation promoter activator (e.g. an amine, such as TEA, DMAP, piperidine, methylpiperidine, or preferably TMEDA) may be added in any suitable quantity, for instance from about 1 to about 50 mol %, based on the quantity of the compound of formula II. Particular embodiments of the invention that may be mentioned include those in which the racemisation promoter activator is used at a concentration of from about 2 to about 30 mol % (e.g. from about 2 or 3 to about 20 or 30 mol %, such as about 10%) based on the quantity of the compound of formula II.

The reagents employed in the process of the invention may be introduced in any feasible, practical order.

Compounds of formula II may be prepared by reduction of a compound of formula III,

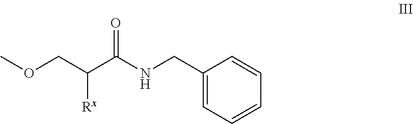

wherein Rx represents —N₃, or another appropriate group that may undergo reduction to form a —NH₂ moiety (e.g. —N(H)—C(H)(R²⁰)R²¹; in which one of R²⁰ and R²¹ represents optionally substituted aryl or optionally substituted heteroaryl (e.g. optionally substituted aryl/heteroaryl) and the other represents hydrogen, optionally substituted C₁₋₁₂ alkyl or optionally substituted aryl or optionally substituted heteroaryl (e.g. optionally substituted aryl/heteroaryl); e.g. R^{x} may represent —N(H)—CH₂-aryl or —N(H)—C(H)-(aryl)/, such as —N(H)—CH₂-phenyl) for instance, under appropriate conditions, e.g. reduction by hydrogenation (or hydrogenolysis), in the presence of hydrogen gas (or a source of hydrogen), in the presence of an appropriate catalyst system (e.g. a precious metal catalyst, such as Pd/C). Advantageously, such an intermediate (e.g. one in which R^{x} is —N₃, and in particular one in which R^{x} is —N(H)—CH₂-phenyl) may be novel and hence of use in this process.

In particular embodiments, compounds of formula II may be prepared by reduction of a compound of formula III, in which R^{x} represents —N₃, or another appropriate group that may undergo reduction to form a —NH₂ moiety (e.g. —N(H)—C(H)(R²⁰)R²¹; in which one of R²⁰ and R²¹ represents optionally substituted aryl/heteroaryl) and the other represents hydrogen, optionally substituted C₁₋₁₂ alkyl or optionally substituted aryl/heteroaryl, under appropriate reducing conditions, as described above.

In particular embodiments of the above processes, the optional substituents are selected from: T³ or C₁₋₁₂ alkyl optionally substituted by one or more substituents selected from T⁴; in which:

T³ and T⁴ are independently selected from halo, —NO₂, —CN, —C(O)₂R^{y1}, —OR^{y2}, —SR^{y3}, —S(O)R^{y4}, —S(O)₂R^{y5}, —N(R^{y6})R^{y7}, —N(R^{y8})C(O)R^{y9}, —N(R^{y10})S(O)₂R^{y11}, —O—P(O)(OR^{y12})(OR^{y13}) or R^{y14};
R^{y1}, R^{y2}, R^{y3}, R^{y6}, R^{y7}, R^{y8}, R^{y9}, R^{y10}, R^{y12} and R^{y13} independently represent hydrogen or C₁₋₆ alkyl optionally substituted by one or more halo atoms;
R^{y4}, R^{y5}, R^{y11} and R^{y14} independently represent C₁₋₆ alkyl optionally substituted by one or more halo atoms.

Compounds of formula II may be prepared in the form of an acid addition salt by reaction of a compound of formula II with an acid of formula VI,

HX VI

wherein X represents a suitable conjugate base e.g. a halide ion or a carboxylate-containing moiety (e.g. a dicarboxylic acid ion (such as oxalic acid, an alkyl (e.g. C₁₋₄ alkyl) dioic acid or an alkenyl (e.g C₂₋₄ alkenyl) dioic acid, particularly maleate, hydrogen maleate, oxalate or, more particularly, hydrogen oxalate)), under appropriate conditions, for example in the presence of a suitable solvent system (e.g. water or an organic solvent system (such as THF, acetone, ethyl ether, methanol/ethyl ether, isopropyl acetate, toluene, methanol methyl-tert-butyl ether, ethanol, isopropyl acetate/2-propanol, heptane or preferably 2-propanol), or mixtures thereof). Maleate and hydrogen maleate salts may in particular be prepared using isopropyl acetate as the solvent system, and oxalate and hydrogen oxalate salts may in particular be prepared using 2-propanol as the solvent system. Advantageously, compounds of formula II may be isolated in their salt forms by this route as crystalline solids with relatively high purity (i.e. with a purity higher than that which would be obtained for the free base form). The salt form products may be optionally isolated and/or purified before further use by, for example, filtration or washing.

In an embodiment of the above process, the compound of formula II is added to a solution of the acid of formula VI, for example a solution of the acid in 2-propanol.

Compounds of formula II may be prepared by converting an acid addition salt of a compound of formula II to the free base form, under appropriate conditions, for example in the presence of a suitable solvent system (e.g. water or an organic solvent system (such as THF, acetone, ethyl ether, methanol/ethyl ether, isopropyl acetate, toluene, methanol methyl-tert-butyl ether, ethanol, isopropyl acetate/2-propanol, heptane or preferably 2-propanol), or mixtures thereof), and in the presence of a base (such as inorganic bases (e.g. Na₂CO₃, NaH, K₂CO₃, K₃PO₄, Cs₂CO₃, t-BuONa or t-BuOK) or an amine (such as triethylamine (TEA), pyridine, dimethylaminopyridine (DMAP), piperidine, methylpiperidine, N,N′-dimethylethylenediamine, 1,4-diazabicyclo[2.2.2]octane (DABCO) or N,N,N′,N′-tetramethylethylenediamine (TMEDA)).

Compounds of formula I may be prepared by:

(a) converting an acid addition salt of a compound of formula II to the free base form, in accordance with the processes described above; followed by
(b) selective enzymatic acylation of the free base product in the presence of an acyl donor, in accordance with the processes described above.

In an embodiment of the above process for the preparation of a compound of formula I, the free base form is isolated and/or purified before the selective enzymatic acylation step is performed.

In an alternative embodiment of the above process for the preparation of a compound of formula I, the steps of:

(a) converting the acid addition salt to the free base form; and
(b) selective enzymatic acylation;
are performed in a “one pot” procedure.

By a “one pot” procedure for this reaction, we mean that, prior to the selective enzymatic acylation step, any undesired acid addition salt of the compound of formula II need not be separated, but rather, in the process of the acylation reaction, the separation of the acid addition salt is circumvented by its conversion to the free base product in the reaction pot.

Compounds of formula III may be prepared by reaction of a compound of formula IV,

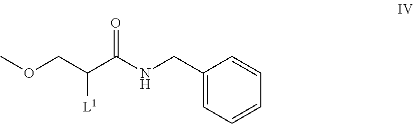

wherein L¹ represents a suitable leaving group, e.g. a sulfonate group or preferably a halo group (e.g. bromo), in the presence of an appropriate amine donor (or group that allows the introduction of the R^{x} moiety), e.g. an azide (e.g. an inorganic metal azide, e.g. sodium azide) or the appropriate amine (e.g. H₂N—C(H)(R²⁰)R²¹, such as benzylamine), under appropriate conditions, for example in the presence of a suitable solvent system (e.g. water or an organic solvent (such as 2-propanol), or mixtures thereof).

Compounds of formula IV in which L¹ represents bromo may be prepared in accordance with the procedures described in international patent application WO 2010/052011. Alternatively and advantageously, such compounds may be prepared by reaction of a compound of formula V,

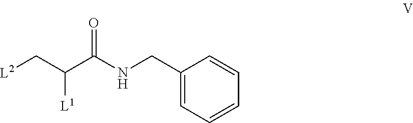

wherein L² represents a suitable leaving group such as one hereinbefore defined by L¹ (e.g. both L¹ and L² may represent bromo), in the presence of a suitable reagent/conditions that promotes the nucleophilic substitution of the L² group with a methoxy group (e.g. regioselectively). For instance, the reaction may be performed in the presence of methanol in an appropriate base (e.g. an alkali metal hydroxide, e.g. sodium hydroxide).

Compounds employed in or produced by the processes described herein (i.e. those involving the process of the invention) may exhibit tautomerism. The process of the invention therefore encompasses the use or production of such compounds in any of their tautomeric forms, or in mixtures of any such forms.

Similarly, the compounds employed in or produced by the processes described herein (i.e. those involving the process of the invention) may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Further, the compounds employed in or produced by the processes described herein (e.g. compounds of formula IIA as hereinbefore defined, which may exist as cis and trans isomers about the imino double bond) may contain double bonds and may thus exist as E (entgegen) and Z (zusammen) geometric isomers about each individual double bond. All such isomers and mixtures thereof are included within the scope of the invention.

Some intermediate compounds disclosed herein may be novel (and useful in the processes described herein). Other intermediate compounds, and derivatives thereof (e.g. protected derivatives), may be commercially available, are known in the literature or may be obtained by conventional synthetic procedures, in accordance with known techniques, from readily available starting materials using appropriate reagents and reaction conditions.

Substituents on compounds of formula I, II, or any relevant intermediate compounds to such compounds (or salts, solvates or derivatives thereof), may be modified one or more times, before, after or during the processes described above by way of methods that are well known to those skilled in the art. Examples of such methods include substitutions, reductions, oxidations, alkylations, acylations, hydrolyses, esterifications, etherifications, halogenations, nitrations, diazotizations or combinations of such methods. Conversions that may be mentioned include —NO₂ to —NH₂ (reduction), —N₃ to —NH₂ (reduction), —N(H)—CH₂-aryl or —N(H)C(H)(aryl)₂ to —NH₂ (reduction e.g. by hydrogenolysis), etc.

It will also be appreciated by those skilled in the art that, in the processes described above, functional groups of intermediate compounds may be, or may need to be, protected by protecting groups.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

In certain embodiments of the invention, the process of the invention may be advantageously performed without separation (e.g. isolation) of any side-products or undesired products.

Where it is stated that the reaction is performed without separation of side-products or undesired products, we include that the product obtained by the process of the invention need not be purified (from any such undesired products).

In this context, we therefore include that the product formed by the process of the invention is not extracted from the reaction mixture, and no separate isolation/purification step need take place (to remove said undesired products).

However, in an embodiment, the compound of formula I produced by the process of the invention may be purified and/or isolated (e.g. from any other products, other than the undesired (S)-enantiomers) under standard conditions, e.g. by chromatography, crystallisation, etc.

The processes described herein may be operated as a batch process or operated as a continuous process and may be conducted on any scale.

### EMBODIMENTS

Embodiments of the invention that may be mentioned include those described above, in the examples below, and in the attached claims. For the avoidance of doubt, such embodiments include the following.

(1) A process for the preparation of a compound of formula I,

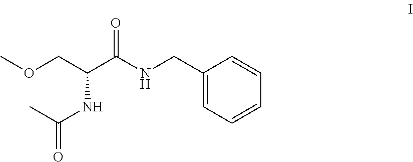 [SMILES: COC[C@@H](NC(C)=O)C(=O)NCc1ccccc1]

which process comprises a selective enzymatic acylation of a compound of formula II,

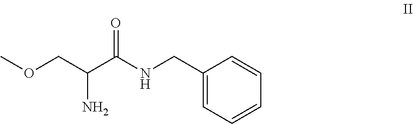

in the presence of an acyl donor.
(2) A process for the preparation of a compound of formula I according to Embodiment 1, wherein the acyl donor is a C₁₋₁₂ alkyl acetate.
(3) A process for the preparation of a compound of formula I according to Embodiment 2, wherein the acyl donor is a branched C₃₋₈ (e.g. C₃₋₄) alkyl acetate.
(4) A process for the preparation of a compound of formula I according to Embodiment 3, wherein the acyl donor is 2-ethylhexyl acetate or, particularly, isopropyl acetate.
(5) A process for the preparation of a compound of formula I according to any one of Embodiments 1 to 4, wherein the process is performed in the presence of a racemisation promoter.
(6) A process for the preparation of a compound of formula I according to Embodiment 5, wherein the racemisation promoter is an aldehyde, ketone or a metal catalyst.
(7) A process for the preparation of a compound of formula I according to Embodiment 6, wherein the racemisation promoter is an aldehyde of formula R¹—CHO, in which R¹ represents optionally substituted aryl or heteroaryl.
(8) A process for the preparation of a compound of formula I according to Embodiment 7, wherein the optional substituents are selected from: T¹ or C₁₋₁₂ alkyl optionally substituted by one or more substituents selected from T²; in which:
T¹ and T² are independently selected from halo, —NO₂, —CN, —C(O)₂R^{x1}, —OR^{x2}, —SR^{x3}, —S(O)R^{x4}, —S(O)₂R^{x5}, —N(R^{x6})R^{x7}, —N(R^{x8})C(O)R^{x9}, —N(R^{x10})S(O)₂R^{x11}, —O—P(O)(OR^{x12})(OR^{x13}) or R^{x14};
R^{x1}, R^{x2}, R^{x3}, R^{x6}, R^{x7}, R^{x8}, R^{x9}, R^{x10}, R^{x12} and R^{x13} independently represent hydrogen or C₁₋₆ alkyl optionally substituted by one or more halo atoms;
R^{x4}, R^{x5}, R^{x11} and R^{x14} independently represent C₁₋₆ alkyl optionally substituted by one or more halo atoms.
(9) A process for the preparation of a compound of formula I according to Embodiment 8, wherein the racemisation promoter is unsubstituted salicylic aldehyde, pyridoxal-5′-phosphate, a dichlorosalicylic aldehyde, 5-nitrosalicylic aldehyde, nitro- or dinitro-benzaldehyde.
(10) A process for the preparation of a compound of formula I according to Embodiment 9, wherein the racemisation promoter is 5-nitrosalicylic aldehyde or 3,5-dichlorosalicylic aldehyde.
(11) A process for the preparation of a compound of formula I according to Embodiment 9, wherein the racemisation promoter is 5-nitrosalicylic aldehyde.
(12) A process for the preparation of a compound of formula I according to any one of Embodiments 5 to 11, wherein the racemisation promoter is used at a concentration of from about 0.1 to about 50 mol % based on the quantity of the compound of formula II.
(13) A process for the preparation of a compound of formula I according to Embodiment 12, wherein the racemisation promoter is used at a concentration of from about 2 to about 20 mol % based on the quantity of the compound of formula
(14) A process for the preparation of a compound of formula I according to any one of Embodiments 5 to 13, wherein the reaction is performed in the presence of a racemisation promoter activator.
(15) A process for the preparation of a compound of formula I according to Embodiment 14, wherein the racemisation promoter activator is an inorganic base or an amine.
(16) A process for the preparation of a compound of formula I according to Embodiment 15, wherein the racemisation promoter activator is Na₂CO₃, triethylamine (TEA), dimethylaminopyridine (DMAP), piperidine, methylpiperidine, or N,N,N′,N′-tetramethylethylenediamine (TMEDA).
(17) A process for the preparation of a compound of formula I according to Embodiment 16, wherein the racemisation promoter activator is TMEDA.
(18) A process for the preparation of a compound of formula I according to any one of Embodiments 14 to 17, wherein the racemisation promoter activator is present at from about 1 to about 50 mol %, based on the quantity of the compound of formula II.
(19) A process for the preparation of a compound of formula I according to Embodiment 18, wherein the racemisation promoter activator is present at from about 2 to about 20 mol %, based on the quantity of the compound of formula II.
(20) A process for the preparation of a compound of formula I according to any one of Embodiments 14 to 17, wherein the racemisation promoter activator is present at from about 2 to about 20 mol %, based on the quantity of the compound of formula II, and the racemisation promoters present at from about 1 to about 3 mol %, based on the quantity of the compound of formula II.
(21) A process for the preparation of a compound of formula I according to any one of Embodiments 1 to 20, wherein the process is performed in the presence of an enantioselective hydrolase enzyme.
(22) A process for the preparation of a compound of formula I according to Embodiment 21, wherein the enzyme is an enantioselective lipase, esterase or protease enzyme.
(23) A process for the preparation of a compound of formula I according to Embodiment 22, wherein the process is performed in the presence of lipase B from Candida Antarctica.
(24) A process for the preparation of a compound of formula I according to any one of Embodiments 1 to 23, wherein the enzyme is immobilised.
(25) A process for the preparation of a compound of formula I according to any one of Embodiments 1 to 23, wherein the enzyme is non-immobilised.
(26) A process for the preparation of a compound of formula I according to any one of Embodiments 1 to 25, wherein the amount of enzyme (plus carrier, if present) employed is from about 10% to about 50% (e.g. from about 20% to about 30%) by weight of the compound of formula II.
(27) A process for the preparation of a compound of formula I according to any one of Embodiments 1 to 26, wherein the process is performed in the presence of a solvent, which solvent is an organic solvent or a mixture of organic solvents.
(28) A process for the preparation of a compound of formula I according to Embodiment 27, wherein the solvent is isopropanol or an acyl donor.
(29) A process for the preparation of a compound of formula I according to Embodiment 28, wherein the solvent is isopropanol or a C₁₋₁₂ alkyl acetate.
(30) A process for the preparation of a compound of formula I according to Embodiment 29, wherein the solvent is isopropyl acetate.
(31) A process for the preparation of a compound of formula I according to Embodiment 29, wherein the solvent is isopropanol.
(32) A process for the preparation of a compound of formula I according to any one of Embodiments 1 to 31, wherein the process is performed in the presence of a co-solvent.
(33) A process for the preparation of a compound of formula I according to Embodiment 32, wherein the co-solvent is DMF, DMAA (N,N-dimethylacetamide), THF, 2-methyl THF, methyl-tert-butyl ether (MTBE), N-methylpyrrolidone (NMP), 2-propanol.
(34) A process for the preparation of a compound of formula I according to Embodiment 33, wherein isopropyl acetate is used as the solvent, THF is the co-solvent and the co-solvent:solvent ratio is from about 10:1 to about 1:10.
(35) A process for the preparation of an acid addition salt of a compound of formula II,

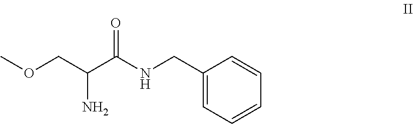

which comprises:
(a) reaction of a compound of formula II with an acid of formula VI,

HX VI

wherein X represents a suitable conjugate base; and
(b) optionally purifying the product of step (a).
(36) A process for the preparation of an acid addition salt of a compound of formula II, as defined in Embodiment 35, wherein the suitable conjugate base is a a dicarboxylic acid ion (such as oxalic acid, an alkyl (e.g. C₁₋₄ alkyl) dioic acid or an alkenyl (e.g. C₂₋₄ alkenyl) dioic acid, particularly maleate, hydrogen maleate, oxalate or, more particularly, hydrogen oxalate ion).
(37) An acid addition salt of a compound of formula II,

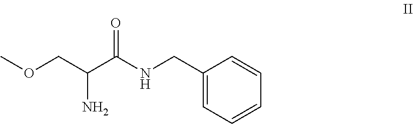

wherein the acid addition salt is a dicarboxylic acid salt (such as an oxalic acid salt, an alkyl (e.g. C₁₋₄ alkyl) dioic acid salt or an alkenyl (e.g C₂₋₄ alkenyl) dioic acid salt).
(38) An acid addition salt according to Embodiment 37, wherein the dicarboxylic acid salt is an oxalate or hydrogen oxalate salt.
(39) A process for the preparation of a compound of formula I,

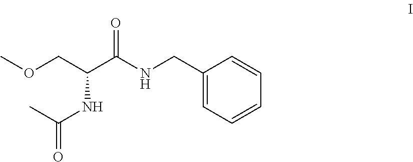 [SMILES: COC[C@@H](NC(C)=O)C(=O)NCc1ccccc1]

which process comprises:
(a) conversion of an acid addition salt of a compound of formula II, as defined in Embodiment 35, to the free base form; followed by
(b) selective enzymatic acylation of the product of step (a) in the presence of an acyl donor.
(40) A process for the preparation of a compound of formula I,

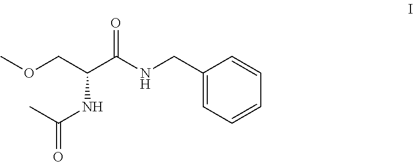 [SMILES: COC[C@@H](NC(C)=O)C(=O)NCc1ccccc1]

which process comprises:
(a) reaction of a compound of formula II, as defined in Embodiment 1, with an acid of formula VI, as defined in Embodiment 35 or Embodiment 36;
(b) optionally purifying the product of step (a);
(c) conversion of the product of step (a) or step (b) to the free base form; and
(d) selective enzymatic acylation of the product of step (c) in the presence of an acyl donor.
(41) A process for the preparation of a compound of formula I, as defined in Embodiment 39 or Embodiment 40, wherein the steps of converting the acid addition salt to the free base form and selective enzymatic acylation are performed in a one pot procedure.
(42) A compound of formula III,

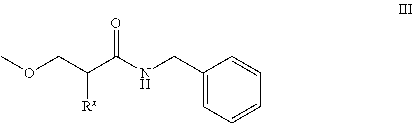

wherein R^{x} represents —N₃ or another group that may undergo reduction to form a —NH₂ moiety.
(43) A compound of formula III according to Embodiment 42, wherein R^{x} represents: —N₃ or —N(H)—C(H)(R²⁰)R²¹; in which one of R²⁰ and R²¹ represents optionally substituted aryl or optionally substituted heteroaryl, and the other represents hydrogen, optionally substituted C₁₋₁₂ alkyl, optionally substituted aryl or optionally substituted heteroaryl.
(44) A compound of formula III according to Embodiment 43, wherein the optional substituents are selected from: T³ or C₁₋₁₂ alkyl optionally substituted by one or more substituents selected from T⁴; in which:
T³ and T⁴ are independently selected from halo, —NO₂, —CN, —C(O)₂R^{y1}, —OR^{y2}, —SR^{y3}, —S(O)R^{y4}, —S(O)₂R^{y5}, —N(R^{y6})R^{y7}, —N(R^{y8})C(O)R^{y9}, —N(R^{y10})S(O)₂R^{y11}, —O—P(O)(OR^{y12})(OR^{y13}) or R^{y14};
R^{y1}, R^{y2}, R^{y3}, R^{y6}, R^{y7}, R^{y8}, R^{y9}, R^{y10}, R^{y12} and R^{y13} independently represent hydrogen or C₁₋₆ alkyl optionally substituted by one or more halo atoms;
R^{y4}, R^{y5}, R^{y11} and R^{y14} independently represent C₁₋₆ alkyl optionally substituted by one or more halo atoms.
(45) A compound of formula III according to Embodiment 44, wherein R^{x} represents —N(H)—CH₂-phenyl or —N(H)—C(H)-(phenyl)₂),
(46) A process for the preparation of a compound of formula II,

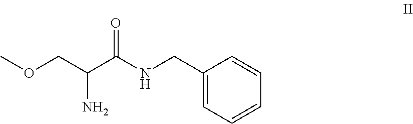

which comprises reduction of a compound of formula III, as defined in any one of Embodiments 42 to 45.
(47) A process for the preparation of an acid addition salt of a compound of formula II,

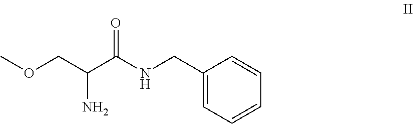

which comprises:
(a) reduction of a compound of formula III, as defined in any one of Embodiments 42 to 45;
(b) reaction of the product of step (a) with an acid of formula VI, as defined in Embodiment 35 or Embodiment 36; and
(c) optionally purifying the product of step (b).
(48) A process for the preparation of a compound of formula I,

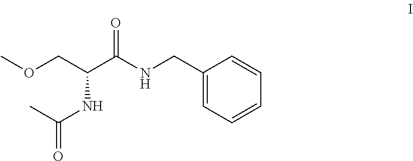 [SMILES: COC[C@@H](NC(C)=O)C(=O)NCc1ccccc1]

which process comprises:
(a) reduction of a compound of formula III, as defined in any one of Embodiments 42 to 45; followed by
(b) selective enzymatic acylation of the product of step (a) in the presence of an acyl donor.
(49) A process for the preparation of a compound of formula I,

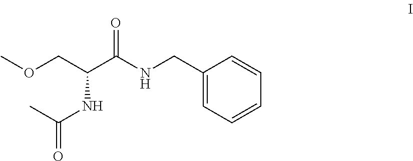 [SMILES: COC[C@@H](NC(C)=O)C(=O)NCc1ccccc1]

which process comprises:
(a) reduction of a compound of formula III, as defined in any one of Embodiments 42 to 45;
(b) reaction of the product of step (a) with an acid of formula VI, as defined in Embodiment 35 or Embodiment 36;
(c) optionally purifying the product of step (b);
(d) conversion of the product of step (b) or step (c) to the free base form; and
(e) selective enzymatic acylation of the product of step (d) in the presence of an acyl donor.
(50) A process for the preparation of a compound of formula I, as defined in Embodiment 49, wherein the steps of converting the acid addition salt to the free base form and selective enzymatic acylation are performed in a one pot procedure.
(51) A process for the preparation of a compound of formula I according to any one of Embodiments 39 to 41 and 48 to 50, wherein the acyl donor is as defined in any one of Embodiments 2 to 4.
(52) A process for the preparation of a compound of formula I according to any one of Embodiments 39 to 41 and 48 to 51, wherein the selective enzymatic acylation step is performed in the presence of a racemisation promoter as defined in any one of Embodiments 5 to 13.
(53) A process for the preparation of a compound of formula I according to Embodiment 52, wherein the selective enzymatic acylation step is performed in the presence of a racemisation promoter activator as defined in any one of Embodiments 14 to 17.
(54) A process for the preparation of a compound of formula I according to Embodiment 53, wherein the racemisation promoter activator is present at from about 1 to about 50 mol %, based on the quantity of the reactant for the selective enzymatic acylation step.
(55) A process for the preparation of a compound of formula I according to Embodiment 54, wherein the racemisation promoter activator is present at from about 2 to about 20 mol %, based on the quantity of the reactant for the selective enzymatic acylation step.
(56) A process for the preparation of a compound of formula I according to Embodiment 53, wherein the racemisation promoter activator is present at from about 2 to about 20 mol %, based on the quantity of the reactant for the selective enzymatic acylation step, and the racemisation promoter is present at from about 1 to about 3 mol %, based on the quantity of the reactant for the selective enzymatic acylation step.
(57) A process for the preparation of a compound of formula I according to any one of Embodiments 39 to 41 and 48 to 56, wherein the selective enzymatic acylation step is performed in the presence of an enantioselective hydrolase enzyme as defined in any one of Embodiments 21 to 26.
(58) A process for the preparation of a compound of formula I according to any one of Embodiments 39 to 41 and 48 to 57, wherein the selective enzymatic acylation step is performed in the presence of a solvent as defined in any one of Embodiments 27 to 31.
(59) A process for the preparation of a compound of formula I according to any one of Embodiments 39 to 41 and 48 to 58, wherein the selective enzymatic acylation step is performed in the presence of a co-solvent as defined in Embodiment 32 or Embodiment 33.
(60) A process for the preparation of a compound of formula I according to Embodiment 59, wherein, in the selective enzymatic acylation step, isopropyl acetate is used as the solvent, THF is the co-solvent and the co-solvent:solvent ratio is from about 10:1 to about 1:10.
(61) A process for preparing a pharmaceutical formulation comprising a compound of formula I, or a salt thereof, which process is characterised in that it includes as a process step a process for the preparation of a compound of formula I according to any one of Embodiments 1 to 34, 39 to 41 and 48 to 60.
(62) A process for preparing a pharmaceutical formulation according to Embodiment 61, wherein the process for the preparation of a compound of formula I is followed by purification of the compound of formula I.
(63) A process for preparing a pharmaceutical formulation according to Embodiment 62, wherein the purification of the compound of formula I is followed by bringing into association the compound of formula I, or a salt thereof, so formed, with one or more pharmaceutically-acceptable excipients, adjuvants, diluents or carriers.
(64) An acid addition salt of a compound of formula II, as defined in Embodiment 35 or, particularly, 36.

In general, the processes described herein, may have the advantage that the compounds of formula I may be produced in a manner that utilises fewer reagents and/or solvents, and/or requires fewer reaction steps (e.g. distinct/separate reaction steps) compared to processes disclosed in the prior art. Processes described herein may also have the advantage that fewer undesired by-products (resultant of undesired side reactions) may be produced, for example, by-products that may be toxic or otherwise dangerous to work with, e.g. explosive.

The processes of the invention may also have the advantage that the compound of formula I is produced in higher yield, in higher purity, in higher selectivity (e.g. higher regioselectivity), in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art. Furthermore, there may be several environmental benefits of the process of the invention.

The following examples are merely illustrative examples of the processes of the invention described herein.

All equipment, reagents and solvents used were standard laboratory equipment, e.g. glassware, heating apparatus and HPLC apparatus.

### Example 1

### Step 1

### N-Benzyl acrylamide

To acrylonitrile, 318.8 g (6 mol) is added sulfuric acid, 235 g (2.4 mol) keeping the temperature below 10° C. Benzyl alcohol, 216.3 g (2 mol) is slowly added below 10° C. The mixture is heated to 60° C. and stirred until the benzyl alcohol is consumed. The mixture is cooled to ca 25° C. and toluene, 173 g, is added. The product solution is washed with 300 g of water followed by 2×100 g of 10% sodium carbonate. The toluene is stripped at reduced pressure and the product 280 g, 86.8% yield, is isolated as an oil that solidifies upon standing.

### Step 2

### N-Benzyl-2,3-dibromo propionamide

N-Benzyl acrylamide, 306.6 g (1.9 mol), 50 g of water and 696 g toluene are added to a reactor. Bromine, 303.9 g is added at 20-30° C. 125 g 20% sodium sulfite is added and the temperature adjusted to 60° C. The water phase is separated, the toluene solution cooled to 20° C., filtered and the filter cake washed with 87 g toluene followed by 200 g water. Drying at 40° C. under reduced pressure afforded 447 g, 73.3% yield of pure product.

### Step 3

### N-Benzyl-2-bromo-3-methoxy propionamide

N-Benzyl-2,3-dibromo propionamide, 211.8 g (0.66 mol) is diluted with 486 g methanol. Sodium hydroxide, 53.1 g (1.33 mol) is added in portions keeping the temperature below 30° C. The mixture is stirred for 2.5 hours and then neutralized with 37% hydrochloric acid. The methanol is stripped under reduced pressure and the residue redissolved in 87 g toluene and washed with 200 g water. After stripping of toluene at reduced pressure, 175 g, 97.6% yield of product is afforded.

### Steps 2 and 3

### N-Benzyl-2-bromo-3-methoxy propionamide

Alternatively, the above product may be prepared by the following method: Bromine, 187.3 g (1.17 mol) was slowly added to a solution of sodium bromide, 102.9 g (1 mol) in 500 ml water under stirring. N-benzyl acryl amide, 161.2 g (1 mol) was then added in portions at such rate as to keep the temperature at max 30° C. Sodium sulfite, 21.4 g (0.17 mol) was added in one portion followed by 200 ml toluene. The mixture was heated to 75-80° C. and the water phase separated. The toluene phase was diluted with 700 ml methanol and then cooled to 16° C. Sodium hydroxide, 80 g (2 mol) was added in portions at such rate as to keep the temperature below 30° C. 37% hydrochloric acid was then added until pH reached 6. Most of the methanol was stripped at reduced pressure and the residual salt slurry was diluted with 300 ml water and 270 ml toluene. The mixture was heated to 60° C., the water phase separated and the toluene phase dried by distillation of 38 g toluene. The toluene phase was further diluted with 305 ml toluene, 10 ml water and 300 ml heptanes. The mixture was cooled to 6° C. and the formed crystal slurry was diluted with 240 ml heptanes. The slurry was further cooled to 2° C., filtered and the filter cake washed with 200 ml toluene/heptanes 50/50 V/V. Drying under vacuum afforded 205.8 g, 76% yield of N-benzyl-2-bromo-3-methoxy propionamide.

### Step 4

### N-Benzyl-2-azido-3-methoxy propionamide

N-Benzyl-2-bromo-3-methoxy propionamide, 227.4 g (83.6 mol) and sodium azide, 55.2 g (0.85 mol) is dissolved in 89% aqueous methanol. The mixture is heated in a closed vessel at 100° C. for 3 hours and then cooled to 50° C. The methanol is stripped under reduced pressure and the residue redissolved in 87 g toluene. The toluene phase is washed with 100 g water and the toluene stripped to leave 183.2 g, 93.6% yield, of the product as a viscous oil.

### Step 5

### N-Benzyl-2-amino-3-methoxy propionamide

N-Benzyl-2-azido-3-methoxy propionamide, 161.8 g (0.69 mol) is dissolved in 395 g methanol. 8.1 g, 3% Pd/C is added and the mixture heated to 30° C. The reactor is pressurized with hydrogen to 5 bar and stirred for 4.5 hours. Formed nitrogen is vented at regular intervals. The catalyst is filtered off and methanol stripped at reduced pressure. The product is redissolved in 87 g toluene and the product extracted to 383 g 22% phosphoric acid. After separation of the toluene phase, sodium hydroxide is added until pH 9 and the product extracted to 131 g toluene. Stripping of the toluene at reduced pressure afforded 133.8 g, 93% yield of the product as light brown syrup.

### Steps 4 and 5

### N-Benzyl-2-amino-3-methoxy propionamide

Alternatively, the above product may be prepared by one the following methods:

(i) N-Benzyl-2-bromo-3-methoxy propionamide, 54.4 g (0.2 mol), benzyl amine, 26 g (0.24 mol), NaHCO₃, 18 g (0.21 mol) and 20 ml water charged to a reactor. The mixture was stirred and heated at 95° C. for 2 h. Water, 60 ml, was added and the water phase separated at 65° C. The residual yellow oil was diluted with 103 ml acetic acid and 1 g 10% Pd/C added. The mixture was heated to 75° C. in an autoclave, pressurized with 5 bar of hydrogen and stirred for 130 minutes. The mixture was cooled to 25° C., filtered and concentrated under vacuum to leave 82.7 g of yellow oil containing 41 g N-Benzyl-2-amino-3-methoxy propionamide corresponding to a yield of 98.5%; and
(ii) N-Benzyl-2-bromo-3-methoxy propionamide (256.0 g; 0.94 mol) was heated under reflux with benzylamine (101.8 g; 0.95 mol) in 2-propanol (500 mL) in the presence of sodium carbonate (60 g; 0.60 mol) for 12 h. Heating was continued for 14 h while distilling slowly the solvent from the top. Residual viscous suspension (405 g) was diluted with 2-propanol (200 mL). Salts were filtered out and washed with 2-propanol (200 mL). Product was obtained as reddish-yellow solution in 2-propanol (94% purity by HPLC).

N-Benzyl-2-(benzylamino)-3-methoxypropionamide solution in 2-propanol (615 g) was reduced in the presence of 5% Pd/C (8 g; 50% water; 2 mmol) at 70-75° C. and 5 bar of hydrogen for 12.5 h. The temperature was increased to 80° C. and the process was continued for 2.5 h. The mixture was filtered after cooling to RT. The cake was washed with 2-propanol (200 mL). Small amount of filtrate was concentrated to pale yellow oily residue. Calculated concentration of the product in solution was about 26% by weight of the residue (93.7% purity by HPLC).

### Step 6

Oxalic acid dihydrate (110.0 g; 0.87 mol) was dissolved in 2-propanol (1000 mL) and solution was heated on water-bath to 60° C. 2-Amino-3-methoxy-N-benzylpropionamide solution from the previous step (647 g; calculated 0.82 mol) was poured into the oxalic acid solution over a few minutes. The mixture turned cloudy, and white light granules began to form. The temperature of the mixture was maintained at 58-63° C. Stirring was continued allowing the mixture to cool to RT in 3 h, then left at RT for overnight. The suspension was filtered, and the filter cake was washed with 2-propanol (3×100 mL). The product (wet weight; 516 g) was dried in vacuum at 40-55° C. affording 225.6 g of 2-amino-N-benzyl-3-methoxypropionamide monooxalate as white solid (assay: 100.04% by titration with NaOH; mp: 122.5-124.5° C.; purity: 99.4% by HPLC; water content: 0.37% by KF titration).

### Step 7

### Synthesis of Lacosamide in Kinetic Resolution Mode

The mixture of 2-amino-N-benzyl-3-methoxypropanamide (1.0 g; 4.8 mmol), CALB (0.24 g) and i-PrOAc (20 ml) was stirred at +35-40° C. for 20 hrs. Enzyme was filtered off, filtrate was washed with 7.5 mL 0.5% HCl, with water (7.5 mL), brine (7.5 mL) and concentrated on rotavapor. Yield 460 mg of yellow-brown oil that crystallized at room temperature. The oil was dissolved in EtOAc (2 ml), diluted with MTBE (5 ml) and left at +4° C. overnight. Solids were filtered, washed with MTBE (2 mL) and dried. Obtained were 120 mg of yellow crystals (purity 97.0% by HPLC, ee 97.4%; α_{D}²⁵=+13.1 (c=1.023, methanol)).

### Synthesis of Lacosamide in Dynamic Kinetic Resolution Mode

Five reaction vials were charged with 2-amino-N-benzyl-3-methoxypropanamide (0.12 g; 0.5 mmol), CALB (25 mg), and iPrOAc (3 ml). Into vials 2 and 3 salicylaldehyde (5 μL and 3 μL respectively) and into vials 4 and 5 pyridoxal-5′-phosphate (12 mg and 6 mg respectively) were added. The vials were stirred at 50-55° C. for 19 hrs. The data given in Table 1 below clearly demonstrate better ee-values at high conversions in the cases when racemiser was added.

TABLE 1

| | Normalized | | Normalized area % | | | |
|---|---|---|---|---|---|---|
| Exper- | area % | | (S)- | (R)- | (R) ee | Racemiser |
| iment | amine | amide | enantiomer | enantiomer | % | Details |
| 1 | 25.5 | 74.5 | 16.5 | 83.5 | 67.0 | No racemiser |
| | | | | | | added |
| 2 | 34.0 | 66.0 | 11.5 | 88.5 | 77.0 | added 10 mol % |
| | | | | | | salicylaldehyde |
| 3 | 28.2 | 71.8 | 9.7 | 90.3 | 80.7 | added 6 mol % |
| | | | | | | salicylaldehyde |
| 4 | 27.5 | 72.5 | 8.8 | 91.2 | 82.3 | added 10 mol % |
| | | | | | | PLP |
| 5 | 24.3 | 75.7 | 10.7 | 89.3 | 78.6 | added 5 mol % |
| | | | | | | PLP |

### Example 2

### Experiment with 3,5-dichlorosalicylic acid as the racemiser in dynamic kinetic resolution mode

Five reaction vials were charged as listed in Table 2 below.

TABLE 2

| Exper- | | iso- | | |
|---|---|---|---|---|
| iment | RacNH₂ | PrOAc | Racemiser | Enzyme |
| 6 | 220 mg | 4.4 mL | 3,5-Dichlorosalicylaldehyde, | CALB, |
| | | | 19 mg 10 mol % | 50 mg |
| | | | | fresh |
| 7 | 210 mg | 4.4 mL | 3,5-Dichlorosalicylaldehyde, | CALB, |
| | | | 19 mg 10 mol % | 50 mg |
| | | | | recycled |
| 8 | 210 mg | 4.4 mL | 5-Nitrosalicylaldehyde, | CALB, |
| | | | 16 mg 10 mol % | 50 mg |
| | | | | recycled |
| 9 | 210 mg | 4.4 mL | 3,5-Dichlorosalicylaldehyde, | CALB, |
| | | | 22 mg 12 mol % | 60 mg |
| | | | | recycled |
| 10 | 200 mg | 4.4 mL | 3,5-Dichlorosalicylaldehyde, | CALB, |
| | | | 35 mg 20 mol % Na₂CO₃, 48 mg | 50 mg |
| | | | | fresh |

RacNH₂ = racemic 2-amino-N-benzyl-3-methoxypropanamide

The results of the experiment in Table 3 below show equal performance of 3,5-dichlorosalicylaldehyde and 5-nitrosalicylaldehyde as racemisers. Additionally (and advantageously), CALB recycled from earlier experiment performed as well as fresh enzyme.

TABLE 3

| | | | Normalized area % | | |
|---|---|---|---|---|---|
| | Normalized area % | | (S)- | (R)- | (R) |
| Experiment | amine | amide | enantiomer | enantiomer | ee % |
| 6 | 12 | 81 | 12.9 | 87.1 | 74.2 |
| 7 | 12 | 80.1 | 13.0 | 87.0 | 73.9 |
| 8 | 9.7 | 74.8 | 11.5 | 88.5 | 77.0 |
| 9 | 10.3 | 77.2 | 11.8 | 88.2 | 76.5 |
| 10 | 11 | 82 | 11.8 | 88.2 | 76.5 |

### Example 3

### Recycling of the Enzyme

To improve the economy of the process it has been demonstrated that the enzyme can be recycled several times as illustrated by the examples below.

It has further been demonstrated that even the mother liquor remaining from isolation of crude Lacosamide by crystallisation can be recycled without significantly reducing the enzyme activity. The mother liquor recycled in this manner contains unreacted amine precursor, dissolved product Lacosamide and racemiser.

Advantageously, the enzyme employed in the process of the invention may be recycled, and hence employed in a further process of the invention (e.g. in a further repetition on another batch). A description of how this might be achieved is explained below.

### Recycling Example of CALB

The solution of racemic 2-amino-N-benzyl-3-methoxypropanamide (5.0 g; 21 mmol) in 50 mL iso-PrOAc also containing 1 g of CALB (from Novozyme; Novozyme 435) and 173 mg (5 mol %) of 5-nitrosalicylaldehyde (NSA) was stirred at 70° C. for 16 h. The amine conversion was 90% determined by HPLC assay. The enzyme was filtered off and washed with 15 mL iso-PrOAc. Combined filtrate and washing solution was concentrated on rotavapor to about ½ volume and the (R)-2-acetamido-N-benzyl-3-methoxypropionamide was crystallised by stirring at 23° C. for 2-3 h. The precipitated product was filtered out and washed with 20 mL iso-PrOAc to afford 2.5 g of crude amide with purity 91.4% HPLC (area %) and ee 94% after the first run. The mother liqueur (containing 0.32 g of starting amine and 1.4 g of amide product by HPLC assay) was recycled with the used enzyme into next run after making the mixture up with 4.7 g of fresh racemic amine (to achieve the starting load of 5 g) and ˜10 mL of iso-PrOAc (to achieve 10% solution).

This way CALB (Novozyme 435) has been recycled 10 times, as shown in Table 4 below. The ratio of amine to amide was typically from 1 to 9 to from 2 to 8 in this series of experiments. The ee of isolated crops was in the range of 96% to 79%. Crude Lacosamide isolated from the first-run mixtures had ee typically of 95-96%. The ee of the crude product from successive runs gradually decreased until 82-79%.

The reaction time had to be prolonged by about 2 times in 10^{th} run compared to the first run to achieve the same conversion that characterises the inactivation rate of the enzyme under these conditions. Deactivation of the enzyme did not affect the enantioselectivity.

After the fourth cycle the mixture turned dark (contained many impurities by HPLC) and the product did not precipitate from the mixture any more. Therefore for the fifth and also for the eighth runs fresh solution of amine in iso-PrOAc was taken.

Totally 31.6 g of crude Lacosamide was isolated (yield 67%). In mother liquors from 4-th, 7-th and 10-th run overall 7.9 g of Lacosamide remained as determined by HPLC.

It is evident to those skilled in the art that recycling of the enzyme can be carried out for example by recycling the filtered-off catalyst into the next batch or by filling the enzyme into a column of suitable size or preferably using a series of columns filled with enzyme of suitable size. In the latter case a new column with a fresh enzyme could be switched in as the last column in the set of columns while the first column is used as a pre-column to protect the downstream columns from any contaminants and to fully utilize the activity of the enzyme.

TABLE 4
Recycling of Novozyme 435 under dynamic kinetic resolution at 70° C.

| | | | | | | | | Recycled | |
|---|---|---|---|---|---|---|---|---|---|
| | | Assay, | | Crude Lacosamide | | | | mother | |
| | | % by | | | HPLC, | | | liqueur | |
| | | HPLC | | | area | yield, | ee, | Amine, | Amide, |
| Run | Time, h | Amine | Amide | Amount g | % | wt % | % | g | g |
| 1 | 16 | 10 | 90 | 2.50 | 91.4 | 48 | 94 | 0.32 | 1.4 |
| 2 | 18 | 23 | 82 | 2.53 | 83.5 | | 90 | 0.80 | 3.0 |
| 3 | 20 | 19 | 81 | 5.00 | 89.0 | | 79 | 0.87 | 2.5 |
| Runs | | | | 10.03 | | 70 | | | |
| 1-3 | | | | | | | | | |
| 4 | 17 | 22 | 57 | — | — | — | — | 0.77 | 4.38¹ |
| 5 | 18 | 21 | 78 | 2.40 | 90.0 | 46 | 96 | 0.76 | 1.57² |
| 6 | 20 | 28 | 69 | 2.52 | 76.0 | | 84 | 0.94 | 2.94 |
| 7 | 22 | 21 | 81 | 5.38 | 84.5 | | 82 | 0.31 | 1.65 |
| Runs | | | | 10.30 | | 75 | | | |
| 5-7 | | | | | | | | | |
| 8 | 26 | 14 | 87 | 2.80 | 90.1 | 54 | 95 | 0.44 | 1.5³ |
| 9 | 28 | 16 | 84 | 4.03 | 79 | | 84 | 0.56 | 2.06 |
| 10 | 30 | 10 | 89 | 4.4 | 79.9 | | 79 | 0.29 | 1.9⁴ |
| Total | | | | 31.56 | | 67 | | | 7.93 |

Notes
¹Amide did not precipitate
²Fresh solution
³Fresh solution. Amide precipitated for overnight
⁴Dark Solution

Recycling Example of CALB (from C-LEcta)

The solution of racemic 2-amino-N-benzyl-3-methoxypropanamide (5.0 g; 21 mmol) in 25 mL iso-PrOAc also containing 1.5 g of CALB (from C-LEcta) and 173 mg (5 mol %) of 5-nitrosalicylaldehyde (NSA) was stirred at 70° C. for 19 h. The amine conversion was 89% determined by HPLC assay. The enzyme was filtered off and washed with 15 mL iso-PrOAc. The combined filtrate was evaporated to dryness and the obtained solid was analyzed by HPLC showing 71.4% content of amide with ee 85.6%.

This way, CALB (from c-LEcta) has been recycled 11 times using fresh reagents in every run, as shown in Table 5 below. The reaction time had to be prolonged by about 1.5 times in 11-th run compared to the first run to achieve the same conversion that characterizes the inactivation rate of the enzyme under these conditions. Deactivation of the enzyme did not affect the enantioselectivity.

After 11 cycles were totally isolated 61.8 of crude product, containing 43.7 g of amide by HPLC assay (yield 77% corrected for purity). The ee of isolated crude products remained in the range from 80% to 90%.

TABLE 5
Recycling of CALB from C-LEcta under dynamic kinetic resolution
at 70° C.

| | | | | Crude solid | | | | |
|---|---|---|---|---|---|---|---|---|
| | | | | | Amide | Yield and purity of | | |
| | | Assay, % by | | | by | Amide | | |
| | Time | HPLC | | Amount | HPLC | Amount | Yield | ee |
| Run | h | Amine | Amide | g | w % | g | % | % |
| 1 | 19 | 11 | 89 | 5.51 | 71.4 | 3.93 | 76 | 85.6 |
| 2 | 21 | 12.3 | 84.5 | 5.64 | 73 | 4.11 | 79 | 89.7 |
| 3 | 21 | 16.6 | 81 | 5.86 | 69 | 4.04 | 78 | 83.4 |
| 4 | 24 | 10.4 | 86.9 | 5.61 | 72.1 | 4.04 | 78 | 79.7 |
| 5 | 26 | 11.8 | 86.8 | 5.44 | 71.9 | 3.91 | 76 | 84.2 |
| 6 | 25 | 12.5 | 87.7 | 5.50 | 71.1 | 3.91 | 76 | 81.3 |
| 7 | 24 | 14.6 | 84.3 | 5.83 | 69.2 | 4.03 | 76 | 85.2 |
| 8 | 24 | — | — | 5.65 | 74.7 | 4.22 | 82 | 88.3 |
| 9 | 27 | — | — | 5.72 | 69.5 | 3.97 | 77 | 85.3 |
| 10 | 25 | 17.1 | 82.1 | 5.60 | 68.9 | 3.86 | 75 | 88.1 |
| 11 | 28 | 14 | 85.9 | 5.45 | 67.5 | 3.68 | 71 | 82.0 |
| Total | | | | 61.8 | | 43.7 | 77 | |

### Example 4

### Purification of Crude Lacosamide

Crude Lacosamide could be purified by recrystallisation from a suitable solvent like ethyl acetate, isopropyl acetate, etc. to bring the ee of the product>99.0%.

Thus, crude Lacosamide from combined 1 to 3 runs in Table 4 above (9 g; purity 88.3% by HPLC area % and 83.2% ee) was dissolved in refluxing EtOAc (90 mL) and cooled then slowly to RT. The crystallization started at <50° C. The slurry was stirred for overnight, filtered and washed with 15 mL EtOAc. Purified Lacosamide (6.0 g; 98.2% purity by HPLC area % and 98.4% ee was achieved.

This recrystallised Lacosamide 3.0 g of was one more time recystallised from 30 mL of EtOAc to obtain 2.2 g of Lacosamide white 98.5% purity by HPLC area % and 99.4% ee.

Crude Lacosamide from run 5 in Table 4 (2.3 g; purity 90% by HPLC area % and 96% ee) was dissolved in refluxing EtOAc (20 mL), cooled then slowly to RT and stirred at RT for 1 h. The precipitate was filtered off and washed with 2 mL EtOAc to afford 1.6 g of purified Lacosamide with 97.5% purity by HPLC area % and 99.6% ee.

Hence, Lacosamide may advantageously be prepared by the procedures described herein, followed by the purification/crystallization techniques described herein. There is hence further provided a method of purification, including increasing ee, of a compound of formula I (e.g. prepared by the processes described herein).

### Example 5

Lacosamide (compound of formula I), e.g. obtained by the procedures disclosed herein, may be formulated into a pharmaceutically acceptable formulation using standard procedures.

For example, there is provided a process for preparing a pharmaceutical formulation comprising Lacosamide of formula I, or a salt thereof, which process is characterised in that it includes as a process step a process as hereinbefore defined. The skilled person will know what such pharmaceutical formulations will comprise/consist of (e.g. a mixture of active ingredient (i.e. Lacosamide or a salt thereof) and pharmaceutically acceptable excipient, adjuvant, diluent and/or carrier).

There is further provided a process for the preparation of a pharmaceutical formulation comprising Lacosamide of formula I (or a salt thereof), which process comprises bringing into association Lacosamide, or a pharmaceutically acceptable salt thereof (which may be formed by a process as hereinbefore described), with (a) pharmaceutically acceptable excipient(s), adjuvant(s), diluent(s) and/or carrier(s).

When a pharmaceutical formulation is referred to herein, it includes a formulation in an appropriate dosage form for intake (e.g. in a tablet form). Hence, any process mentioned herein that relates to a process for the preparation of a pharmaceutical formulation comprising Lacosamide, or a salt thereof, may further comprise an appropriate conversion to the appropriate dosage form (and/or appropriate packaging of the dosage form).

### Example 6

### Racemisation Promoter Activation

The racemisation of 2-amino-N-benzyl-3-methoxypropionamide (S-amine) took place with satisfactory rate at 10 mol % concentrations of racemisation promoter (5-nitrosalicylic aldehyde, NSA) using racemic 2-amino-N-benzyl-3-methoxypropionamide.

Addition of 10 mol % of TMEDA (calculated with respect to the racemic 2-amino-N-benzyl-3-methoxypropionamide) allowed the reduction of the amount of NSA from 10 mol % to 1 mol % (calculated with respect to the racemic 2-amino-N-benzyl-3-methoxypropionamide) while retaining the same racemisation rate.

The effects from different racemisation promoter activators on the racemisation rate of (S)-2-amino-N-benzyl-3-methoxypropionamide are summarised in Table 6 below. Triethylamine (TEA) and tetramethylethylenediamine (TMEDA) were superior compared to other bases tested.

TABLE 6
Results of racemisation of S-amine (1% solution in iPrOAc) at
69-72° C. at different racemisation activator concentration
and in the presence of base. Starting S-amine had 80.6% ee.

| | | Enantiomeric | | | |
|---|---|---|---|---|---|
| Exp. | Time, | ratio by | ee % of | Racemiser, | Base, |
| No. | h | HPLC, area % | amine | mol % | mol % |
| 6.01 | 3 | 15.6/84.3 | 68.8 | — | TEA, |
| | | | | | 5 mol % |
| 6.02 | 1 | 33/67 | 34.0 | NSA, | — |
| | 3 | 45.2/54.7 | 9.5 | 10 mol % | |
| 6.03 | 1 | 15.5/84.5 | 69 | NSA | — |
| | 3 | 25.6/68 | 45 | 1 mol % | |
| 6.04 | 1 | 17.1/78.7 | 64.3 | NSA | TEA, |
| | 3 | 27.5/66.8 | 41.7 | 1 mol % | 10 mol % |
| 6.05 | 1 | 19.5/78 | 60 | NSA | — |
| | 3 | 33.5/61 | 29 | 2 mol % | |
| 6.06 | 1 | 22.3/74.5 | 54 | NSA | TEA, |
| | 3 | 37.3/57.1 | 21 | 2 mol % | 5 mol % |
| 6.07 | 1 | 20.1/76.3 | 58 | NSA | DMAP, |
| | 3 | 32.5/62 | 31 | 2 mol % | 5 mol % |
| 6.08 | 1 | 23.8/72.3 | 50.5 | NSA | Na₂CO₃, |
| | 3 | 36.6/58.1 | 22.7 | 2 mol % | 10 mol % |
| 6.09 | 1 | 28.6/67.6 | 40.5 | NSA | Piperidine, |
| | 3 | 41/53.5 | 13.2 | 2 mol % | 5 mol % |
| 6.10 | 1 | 20.7/74.4 | 56.5 | NSA | Methylpiperidine, |
| | 3 | 35/60 | 26 | 2 mol % | 5 mol % |
| 6.11 | 1 | 19.5/75 | 58.8 | NSA | TEA, |
| | 3 | 32.4/61.9 | 31 | 2 mol % | 30 mol % |
| 6.12 | 1 | 19/75 | 59 | NSA | TEA, |
| | 3 | 32.4/61.9 | 31 | 2 mol % | 20 mol % |
| 6.13 | 3 | 45.9/54 | 8.1 | NSA | TEA, |
| | | | | 3 mol % | 5 mol % |
| 6.14 | 0.5 | 37/63 | 26.2 | NSA | TMEDA. |
| | 1 | 43/57 | 14.5 | 1 mol % | 10 vol % |

The experiments have shown that the enzymatic reaction is accelerated under basic conditions. This has been demonstrated through improved reaction rates resulting from the addition of basic compounds (such as TEA and Na₂CO₃), but also in running the process in more concentrated solutions of racemic 2-amino-N-benzyl-3-methoxypropionamide which is a basic compound in its nature as well. Unfortunately the accelerating effect of racemic 2-amino-N-benzyl-3-methoxypropionamide ceases at the end of the reaction due to the lowering of its concentration. For that reason, addition of a suitable base is preferred in order to maintain a higher reaction rate at high conversions.

In another set of experiments racemisation of S-amine (ee-80.6%) with NSA (1-2 mol %) in the presence of 10-30% (volume %) of triethylamine or 5-10 mol % TMEDA was tested. Results are depicted in Table 7.

TABLE 7
Results of racemisation of S-amine (1% solution
in iPrOAc) at 69-72° C. in the presence of
NSA and TEA (or TMEDA). Starting S-amine had 80.6% ee.

| | | Enantiomeric | | | |
|---|---|---|---|---|---|
| | Time, | ratio by HPLC, | ee % of | NSA | TEA, |
| Exp. No. | h | area % | amine | mol % | vol % |
| 7.1 | 1 | 13.2/86.8 | 73.6 | 1 | — |
| | 3 | 21.7/78.3 | 56.5 | | |
| 7.2 | 0.5 | 20/80 | 60 | 1 | 10 |
| | 1 | 26.7/73.3 | 46.6 | | |
| | 2 | 35.8/64.2 | 28.5 | | |
| | 3 | 41.2/58.8 | 17.6 | | |
| 7.3 | 0.5 | 21.3/78.7 | 57.3 | 1 | 20 |
| | 1 | 28.2/71.8 | 43.6 | | |
| | 2 | 36.4/63.6 | 27.1 | | |
| 7.4 | 0.5 | 24.2/75.8 | 51.7 | 1 | 30 |
| | 1 | 31.1/68.9 | 37.9 | | |
| | 2 | 39.3/60.7 | 21.3 | | |
| | 3 | 43/57 | 13.9 | | |
| 7.5 | 0.5 | 36.8/63.2 | 26.3 | 2 | 10 |
| | 1 | 40.9/59.1 | 18.2 | | |
| | 2 | 46.6/53.4 | 6.9 | | |
| | 3 | 49.7/50.3 | 0.6 | | |
| 7.6 | 1 | 13/87 | 74 | 2 | — |
| | 2 | 20/73 | 57 | | |
| 7.7 | 1 | 19.5/78 | 60 | 2 | — |
| | 3 | 33.5/61 | 29 | | |
| 7.8 | 0.5 | 36.9/63.1 | 26.2 | 1 | TMEDA |
| | 1 | 42.7/57.3 | 14.5 | | 10 mol % |
| 7.9 | 0.5 | 15.6/84.4 | 69 | 0.5 | TMEDA |
| | 1 | 24.1/75.9 | 52 | | 5 mol % |
| | 2 | 26.9/73.1 | 46 | | |
| | 3 | 33.9/66.1 | 32 | | |

From the data in Table 7 it is evident that TMEDA is superior to TEA

### Example 7

Dynamic kinetic resolution studies in which a co-solvent has been introduced have also been conducted. THF and DMF were tested as co-solvents.

TABLE 8
Dynamic kinetic resolution of racemic 2-amino-N-benzyl-3-methoxypropionamide (RA) in iPrOAc
(10% and 20% solution) with CalB in the presence of NSA (1 mol %) and TMEDA

| | | Amine/amide | | | | |
|---|---|---|---|---|---|---|
| | | ratio by | | | Co | |
| Exp. | Time, | HPLC, | ee % of | TMEDA, | solvent:iPrOAc | |
| No. | h | area % | amide | % | vol/vol | Notes |
| 8.1 | 1.5 | 76/24 | — | 10 vol % | DMF | RA 10% |
| | 24 | 23/77 | | | 1:2 | solution |
| 8.2 | 1.5 | 77.6/22.4 | 86.2 | 80 mol % | THF | RA 20% |
| | 21 | 26/74 | 89.5 | (10 vol %) | 1:2 | solution |
| | 28 | 20/80 | | | | |
| | 45 | 15/85 | | | | |
| 8.3 | 2.5 | 76/24 | — | 80 mol % | THF | RA 10% |
| | 21 | 26/74 | | (5 vol %) | 1:2 | solution |
| | 45 | 19/81 | | | | |
| 8.4 | 1.5 | 99/1 | — | 80 mol % | DMF | RA 20% |
| | 21 | 78/22 | | (10 vol %) | 1:2 | solution |
| 8.5 | 21 | 74/26 | — | 80 mol % | DMF | RA 10% |
| | | | | (5 vol %) | 1:2 | solution |
| 8.6 | 1.5 | 61/38 | 85.4 | 80 mol % | — | RA 20% |
| | 21 | 9.7/90.3 | | (10 vol %) | | solution. |
| | | | | | | Amide |
| | | | | | | precipitated |
| 8.7 | 1.5 | 69.4/30.6 | 88.8 | 50 mol % | THF | RA 20% |
| | 3 | 57/43 | | (6.2 vol %) | 1:3 | solution |
| | 21 | 12.8/87.2 | | | | |
| 8.8 | 1.5 | 65.5/34.5 | 87.4 | 30 mol % | — | RA 20% |
| | 3 | 53/47 | | (5 vol %) | | solution |
| | 21 | 9.8/90.2 | | | | |

Although addition of THF slightly decreased the reaction rate, it allowed the carrying out of the process with highly pure racemic 2-amino-N-benzyl-3-methoxypropionamide in 20% (w/w) solution and the co-solvent is easy to remove by distillation.

### Abbreviations

CALB lipase B from Candida antarctica
DABCO 1,4-diazabicyclo[2.2.2]octane

### DMAA N,N-dimethylacetamide

DMAP dimethylaminopyridine
DMF dimethylformamide
ee enantiomeric excess
h hours
HPLC high performance liquid chromatography
MTBE methyl-tert-butyl ether

### NMP N-methylpyrrolidone

NSA 5-nitrosalicylic aldehyde
RA racemic 2-amino-N-benzyl-3-methoxypropionamide
RT room temperature
TEA triethylamine
THF tetrahydrofuran
TMEDA tetramethylethylenediamine

1. A process for the preparation of Lacosamide (formula I): 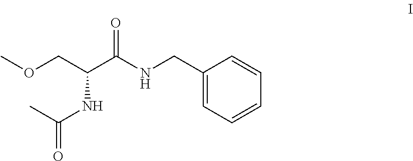 [SMILES: COC[C@@H](NC(C)=O)C(=O)NCc1ccccc1]
which process comprises a selective enzymatic acylation, in the presence of an acyl donor, of a precursor of formula II, 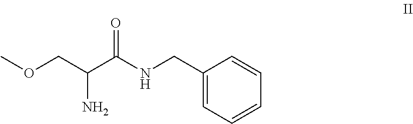
further characterised in that the reaction is performed in the presence of a racemisation promoter.

2. A process as claimed in claim 1, wherein the acyl donor is C₁₋₈ alkyl acetate.

3. A process as claimed in claim 1, wherein the reaction is performed in the presence of an enantioselective hydrolase.

4. A process as claimed in claim 3, wherein the enzyme is recovered and is optionally reused.

5. The process of claim 1 wherein the racemisation promoter is an aldehyde, ketone or metal catalyst.

6. A process as claimed in claim 5, wherein the racemisation promoter is an aldehyde that is R¹—CHO, in which R¹ represents optionally substituted aryl or heteroaryl.

7. A process as claimed claim 6, wherein the aldehyde is selected from unsubstituted salicylic aldehyde, pyridoxal-5′-phosphate, dichlorosalicylic aldehyde, 5-nitrosalicylic aldehyde, nitro-benzaldehyde or dinitro-benzaldehyde.

8. The process of claim 1 wherein the process takes place at a temperature from room temperature to about 100° C.

9. The process of claim 1 wherein the racemisation promoter is employed in from about 0.1 to about 50 mol %, based on the quantity of the compound of formula II.

10. The process of claim 1 wherein the enzyme is employed in from about 10 to about 50% by weight of the compound of formula II.

11. The process of claim 1 wherein the reaction is performed in the presence of a racemisation promoter activator.

12. A process as claimed in claim 11, wherein the racemisation promoter activator is an inorganic base or an amine.

13. The process of claim 1 wherein the reaction is performed in the presence of a co-solvent.

14. A process as claimed in claim 13, wherein the co-solvent is tetrahydrofuran.

15. A process for the preparation of a compound of formula II, 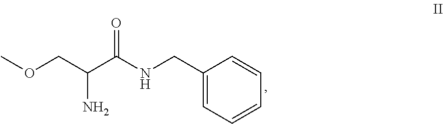
which comprises reduction of a compound of formula III, 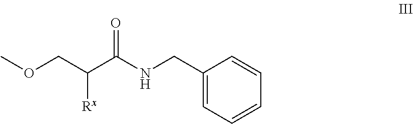
wherein R^{x} represents —N₃ or —N(H)—C(H)(R²⁰)R²¹; in which one of R²⁰ and R²¹ represents optionally substituted aryl or optionally substituted heteroaryl and the other represents hydrogen, optionally substituted C₁₋₁₂ alkyl, optionally substituted aryl or optionally substituted heteroaryl.

16. A compound of formula III 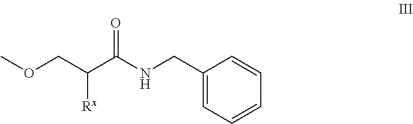
wherein R^{x} represents —N₃ or —N(H)—C(H)(R²⁰)R²¹; in which one of R²⁰ and R²¹ represents optionally substituted aryl or optionally substituted heteroaryl and the other represents hydrogen, optionally substituted C₁₋₁₂ alkyl, optionally substituted aryl or optionally substituted heteroaryl.

17. A process for preparing a pharmaceutical formulation comprising a compound of formula I, or a salt thereof, 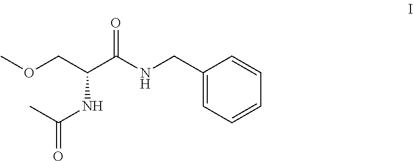 [SMILES: COC[C@@H](NC(C)=O)C(=O)NCc1ccccc1]
which process is characterised in that it includes as a process step a process as claimed in claim 1, optionally followed by purification of the compound of formula I (including optical purification) optionally followed by bringing into association the compound of formula I (or a salt thereof) so formed, with (a) pharmaceutically-acceptable excipient(s), adjuvant(s), diluent(s) or carrier(s)).

18. (canceled)

19. A process for the preparation of an acid addition salt of a compound of formula II, 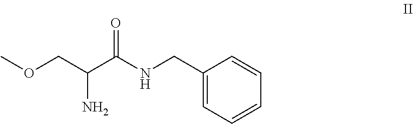
which comprises:
(a) reduction of a compound of formula III 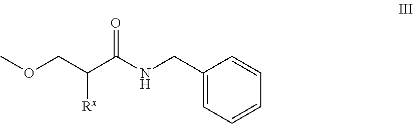
wherein R^{x} represents —N₃ or —N(H)—C(H)(R²⁰)R²¹; in which one of R²⁰ and R²¹ represents optionally substituted aryl or optionally substituted heteroaryl and the other represents hydrogen, optionally substituted C₁₋₁₂ alkyl, optionally substituted aryl or optionally substituted heteroaryl;
(b) reaction of the product of step (a) with an acid of formula VI,
HX VI
wherein X represents a suitable conjugate base; and
(c) optionally purifying the product of step (b).

20. A process for the preparation of an acid addition salt of a compound of formula II, according to claim 19, wherein the acid addition salt is a hydrogen oxalate salt.

21. The process of claim 2 where the acyl donor is isopropyl acetate.

22. The process of claim 3 where the enantioselective hydrolase is a lipase.

23. The process of claim 22 where the lipase is lipase B from Candida antarctica).

24. The process of claim 7 where the aldehyde is selected from 3,5-dichlorosalicylic aldehyde, 2-nitro-benzaldehyde, 4-nitro-benzaldehyde, or 2,4-dinitro-benzaldehyde.

25. The process of claim 12 wherein the inorganic base is Na₂CO₃.

26. The process of claim 12 wherein the amine is selected from triethylamine, dimethylaminopyridine, piperidine, methylpiperidine, or N,N,N′,N′-tetramethyl-ethylenediamine.

27. The process of claim 15 wherein Rx is selected from —N(H)—CH₂-phenyl, or —N(H)—C(H)-(phenyl)₂.

28. A process for the preparation of a compound of formula III 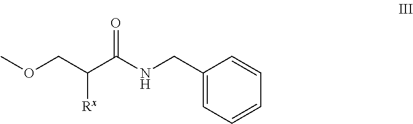
wherein R^{x} represents —N₃ or —N(H)—C(H)(R²⁰)R²¹; in which one of R²⁰ and R²¹ represents optionally substituted aryl or optionally substituted heteroaryl and the other represents hydrogen, optionally substituted C₁₋₁₂ alkyl, optionally substituted aryl or optionally substituted heteroaryl,
which process comprises reaction of a compound of formula IV, 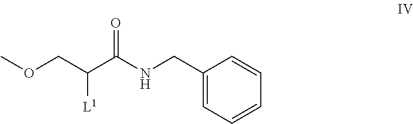
wherein L¹ represents a suitable leaving group, in the presence of an appropriate amine donor or group that allows introduction of the R^{x} moiety.

29. The process of claim 28 wherein the amine donor or group is an azide or a compound of the formula H₂N—C(H)(R²⁰)R²¹.
